(19) United States
(12) Patent Application Publication
(10) Pub. No.: US 2024/0058369 A1
(43) Pub. Date: Feb. 22, 2024

(54) METHOD FOR TREATING OCULAR SURFACE DISEASES

(71) Applicant: DREAMHAWK VISION BIOTECH, INC., Kaohsiung City, Taiwan (CN)

(72) Inventors: Suh-Hang JUO, Kaohsiung City, Taiwan (CN); Chung-Ling LIANG, Kaohsiung City, Taiwan (CN)

(21) Appl. No.: 18/260,773

(22) PCT Filed: Aug. 9, 2021
(86) PCT No.: PCT/CN2021/111575

§ 371 Date: Jul. 7, 2023

Related U.S. Application Data
(60) Provisional application No. 63/134,599, filed on Jan. 7, 2021.

(51) Int. Cl. A61K 31/7105; A61P 27/02

(52) U.S. Cl. CPC A61K 31/7105; A61P 27/02

(57) ABSTRACT

Provided a method of treating ocular surface damage caused by an eye disease or eye injury like dry eye disease, chemical or physical injury, infection, neurosensory abnormalities and unspecified etiologies in a subject, comprising administering to said subject a pharmaceutical composition comprising a therapeutically effective amount of microRNA-328 antagonist.

### FIELD OF THE INVENTION

The present invention relates to a method for treating ocular surface damage caused by an eye disease or eye injury like dry eye disease, chemical or physical injury, infection, neurosensory abnormalities and unspecified etiologies in a subject, comprising administering miRNA-328 antagonist to the subject.

### BACKGROUND OF THE INVENTION

The surface of the eye consists of the cornea and the conjunctiva. Damage to the ocular surface can cause pain, redness, poor vision, and ultimately can lead to permanent corneal or conjunctival injury.

A number of eye diseases or eye injuries may cause ocular surface damage. Dry eye disease is the most common ocular surface disease. It is a multifactorial disease of the tears and ocular surface that results in symptoms of discomfort, visual disturbance and tear film instability. The International Dry Eye Workshop (DEWS) organized by the Tear Film and Ocular Surface Society (TFOS) reported “TFOS DEWS II Definition and Classification” (Ocul Surf. 2017 July; 15(3): 276-283) and provided a definition of the dry eye disease herein. Environmental factors are also often implicated in dry eye including exposure to pollutants, ultraviolet (UV) radiation and ozone as well as the chronic use of preserved eyedrops such as in the treatment of glaucoma. These factors increase oxidative stress and ocular surface inflammation, which contribute to the development of dry eye disease. Recently, Meibomian gland dysfunction has been proposed to be another major risk factor for dry eye disease. Typical symptoms of dry eye disease are dryness, burning and a sandy-gritty eye irritation that gets worse as the day goes on. Both eyes usually are affected. Having dry eyes for a while can lead to tiny abrasions on the surface of the eyes, corneal erosion, and punctate keratopathy. In advanced cases, the epithelium undergoes pathologic changes, namely squamous metaplasia and loss of goblet cells.

Chemical injury including alkali and acid injuries to the eyes produces extensive damage to the ocular surface. Such damages may result in permanent visual impairment if the cornea repairment is incomplete. Physical injury ranging from foreign body, trauma, to pieces of metal can lead to different degree of damage to ocular surface. Ocular infection can result in corneal erosion. If the re-growth of corneal epithelium does not take place, the corneal erosion may develop to corneal ulceration. Untreated corneal ulcers can lead to severe vision loss.

Neurotrophic keratitis, also known as neurotrophic keratopathy (NK) is characterized by a reduction or absence of corneal sensitivity because the corneal nerve innervation is impaired. The loss of corneal sensation may result in epithelial keratopathy, epithelial defect, stromal ulceration, and eventually corneal perforation. It is a rare disease and the estimated prevalence is less than 5/10,000 individuals. The etiology of NK includes, but not limited to, herpes keratitis (zoster and simplex), chronic use of topical medications containing benzalkonium chloride (BAK), chemical and physical burns, contact lens abuse, corneal surgery such as laser in situ keratomileusis (LASIK) etc. Human nerve growth factor (NGF) has been shown to effectively treat patients with NK. Cenegermin (Oxervate™) that contains recombinant human nerve growth factor (rhNGF) is the first approved topical medication for NK for the treatment of neurotrophic keratitis in the United States on Aug. 22, 2018.

The goblet cells are confined to the conjunctival epithelium. The main function of goblet cells is to produce and secrete mucins, which hydrate and lubricate ocular surfaces. Mucins are highly glycosylated glycoproteins. Mucins are classified into two different types: membrane-spanning mucins and secretory mucins. MUC5AC is one of the most studied mucins, and it is a large gel-forming secretory mucin found in conjunctiva. A reduced number of goblet cells in the conjunctiva has been demonstrated in the patients with dry eye disease. Furthermore, the use of contact lens changes the goblet cell density.

MicroRNAs (miRNAs) are noncoding, single-stranded RNA molecules of about 21-23 nucleotides in length. In animals, a mature miRNA is complementary to the 3′ untranslated region (UTR) of one or more messenger RNAs (mRNAs). The annealing of a miRNA to its target mRNA causes an inhibition of protein translation, and/or cleavage of the mRNA. It has been previously reported that microRNA-328 (miR-328) is a risk factor for myopia (Chen et al., Invest. Ophthalmol. Vis. Sci., 53:2732-2739, 2012) and anti-miR-328 oligonucleotides were designed to treat myopia (Juo et al., U.S. Ser. No. 10/179,913B2, 2019).

### SUMMARY OF THE INVENTION

The present invention relates to a method of treating ocular surface damage caused by an eye disease or eye injury like dry eye disease, chemical or physical injury, infection, neurosensory abnormalities and unspecified etiologies, in a subject, comprising administering to said subject a pharmaceutical composition comprising a therapeutically effective amount of microRNA-238 antagonist.

### BRIEF DESCRIPTION OF THE DRAWINGS

FIG. 1 shows that miR-328 expression levels have a dose-dependent increase in the rabbit corneal cell line, SIRC, treated with different concentrations of BAC. The data, mean±SEM, were from 3 independent experiments.

FIG. 2 shows that anti-miR-328 dose-dependently increases NGF in the rabbit corneal cell line, SIRC, after 10-min exposure to BAC. The data, mean±SEM, were from 3 independent experiments.

FIG. 3 shows the representative images of rabbit corneal staining after phosphate-buffered saline (PBS) or anti-miR-328 treatment. PBS was used as a negative control in this study. Dry eye disease was induced by BAC from Day 0 to Day 21. The treatment of PBS (upper panel) or anti-miR-328 (lower panel) eye drops started on Day 8 to Day 21. The green fluorescent light is the corneal stain that indicates corneal damage due to dry eye disease. On Day 21, there was no corneal staining in the rabbits treated with anti-miR-328, while there was still corneal staining in the PBS group.

FIG. 4 shows representative images of H&E staining of corneal sections from the rabbits. Rabbits with dry eye disease were treated with PBS or anti-miR-328, and the normal rabbits were not treated with any eye drops. (A) The representative H&E staining of corneas in normal, PBS and anti-miR-328 groups. The corneal epithelium in the PBS-treated eye was thinner and more destructive, while the epithelial layer in the anti-miR-328-treated eye was thicker and more intact. There was no statistical difference in stroma among normal, PBS-treated and anti-miR-328-treated eyes. Scale bar=50 μm; (B) the scatter plots show the differences of epithelial and stromal thickness among three groups.

FIG. 5 shows representative images of rabbit apoptotic corneal cells detected by the TUNEL assay. Rabbits with dry eye disease were treated with PBS or anti-miR-328, and the normal rabbits were not treated with any eye drops. (A) The apoptotic cells have brown staining by the TUNEL assay. The anti-miR-328-treated eyes had fewer apoptotic cells in both corneal epithelial and stromal layers than PBS-treated eye, while the normal eyes barely had apoptotic cells. Scale bar=100 μm; (B) the scatter plots show the differences of the apoptotic cells among three groups.

FIG. 6 shows the representative images of the orifices of Meibomian glands in the rabbits. Hyperkeratosis (arrow) in the orifice of Meibomian glands was shown in the PBS-treated rabbit eye but not in the anti-miR-328-treated rabbit eye. scale bar=100 μm.

FIG. 7 shows representative images of dose-dependent effect of anti-miR-328 on the mice with dry eye disease. Mouse eyes were induced to have dry eye disease by BAC and simultaneously treated with anti-miR-328 (with a 10-min interval) twice per day for 14 days. The representative pictures were taken on day 14. The data show the dose of 160 μM has the best therapeutic effect.

FIG. 8 shows that anti-miR-328 repairs corneal abrasion in mouse eyes. The corneal abrasion was generated by Algerbrush on both eyes. Then the left eye was treated with PBS and right eye was treated with anti-miR-328 (160 μM). The eye drops were instilled twice per day starting from the same day when corneal abrasion was generated.

### DETAILED DESCRIPTION OF THE INVENTION

The present invention relates to a method of treating ocular surface damage caused by an eye disease or eye injury like dry eye disease, chemical or physical injury, infection, neurosensory abnormalities and unspecified etiologies in a subject, comprising administering to said subject a pharmaceutical composition comprising a therapeutically effective amount of microRNA-238 antagonist.

The term “subject” as used herein refers to an animal, especially a mammal. In a preferred embodiment, the term “subject” refers to a “human.” Unless otherwise specified, “a” or “an” means “one or more”.

The terms “therapeutically effective” is intended to qualify the amount of each agent which will achieve the goal of decreasing disease severity while avoiding adverse side effects such as those typically associated with alternative therapies.

In one aspect of the invention, the pharmaceutical composition further comprises a pharmaceutically acceptable salt, carrier, adjuvant or excipient.

In another aspect of the invention, the microRNA-238 antagonist is an anti-miR-328 oligonucleotide comprises an oligonucleotide sequence complementary to miR-238, or a precursor thereof.

In one embodiment, anti-sense miR-328 oligonucleotides (15-22 nucleotides in length) are designed according to mature human miR-328 sequence (SEQ ID NO: 1, CUGGCCCUCUCUGCCCUUCCGU). The sequences of anti-miR-328 oligonucleotides which is 15-22 nucleotides in length are shown in Table 1. These anti-miR-328 oligonucleotides were disclosed in a previous patent, U.S. Ser. No. 10/179,913B2, which is incorporated entirely herein by reference.

TABLE 1
Anti-miR-328 oligonucleotides sequence

| SEQ | | |
|---|---|---|
| ID | | |
| NO: | Name | Sequence |
| 2 | Anti-miR-328 15mer | 5′-GGGCAGAGAGGGCCA-3′ |
| 3 | Anti-miR-328 16mer | 5′-AGGGCAGAGAGGGCCA-3′ |
| 4 | Anti-miR-328 17mer | 5′-AAGGGCAGAGAGGGCCA-3′ |
| 5 | Anti-miR-328 18mer | S′-GAAGGGCAGAGAGGGCCA-3′ |
| 6 | Anti-miR-328 19mer | 5′-GGAAGGGCAGAGAGGGCCA-3′ |
| 7 | Anti-miR-328 20mer | 5′-CGGAAGGGCAGAGAGGGCCA-3′ |
| 8 | Anti-miR-328 21mer | 5′-ACGGAAGGGCAGAGAGGGCCA-3′ |
| 9 | Anti-miR-328 22mer | 5′-ACGGAAGGGCAGAGAGGGCCAG-3′ |

In one embodiment, the anti-miR-328 oligonucleotide ranges from 15 to 22 nucleotides in length. In another embodiment, the anti-miR-328 oligonucleotide is 16 or 17 nucleotides in length. In a preferred embodiment, the anti-miR-328 oligonucleotide is consisted of SEQ ID NO: 3 or SEQ ID NO:4. In a more preferred embodiment, the anti-miR-328 oligonucleotide is consisted of SEQ ID NO: 3.

The present invention is directed to a pharmaceutical composition comprising miRNA-328 anti-sense oligonucleotides and a pharmaceutically acceptable carrier. A form for treating ocular diseases is a topical solution or a topical ointment. In one embodiment, the pharmaceutical composition is administered to the eye topically. In another embodiment, the pharmaceutical composition is administered in the form of eye drops.

### EXAMPLES

The examples below are non-limiting and are merely representative of various aspects and features of the present invention.

### Example 1. The Effects of Anti-miR-328 Oligonucleotides Based on In Vitro Studies

The Rabbit Cornea (SIRC) cell line was cultured in DMEM supplemented with 10% fetal bovine serum (FBS) and 100 U/mL of penicillin at 37° C. in an atmosphere of 5% C02. The cells were exposed to benzalkonium chloride for 10 minutes and then the culture medium was replaced with fresh media. For measuring miR-328 level, cells were incubated for a further 24 hours and then harvested for RNA extraction. For measuring NGF, anti-miR-328 was treated to the cells for 12 h, after 10-min exposure to BAC.

### Example 2. In Vivo Study of Mouse Model to Evaluate the Efficacy of Anti-miR-328 Oligonucleotides in Treating Dry Eye Disease

Mouse Model of Dry Eye Disease

Benzalkonium chloride (BAC) is the most commonly used eye drop preservative. However, BAC has toxicity effects on the eyes, including conjunctival inflammation and fibrosis, tear film instability, corneal cytotoxicity, and anterior chamber inflammation. Therefore, a high concentration of BAC has been widely used to induce dry eye disease in the animal models due to its toxic effects. First, C57BL6 mice were randomly divided into the anti-miR-328 and PBS saline groups. Notably, PBS is the solvent to make anti-miR-328 eye drops. 0.2% BAC of 5 μL was instilled to both eyes of a C57BL6 mouse once per day for 7 days to induce dry eye disease. On Day 8, each eye started to receive either 5 μL PBS saline solution or anti-miR-328 (10 μM) eye drops daily for 2 weeks, while BAC was still instilled to the eyes for these 2 weeks. The order of treatment in these 2 weeks is ocular instillation of PBS or anti-miR-328 first, and in approximately 10 min BAC instillation. This strategy is to simulate that the cause of dry eye cannot be removed while a patient receives anti-dry eye treatment.

Outcome Assessment

The therapeutic effect of the anti-miR-328 was evaluated in the end of 2-week PBS/anti-miR-328 treatment. Clinical observation was conducted daily and fluorescein staining was performed weekly. For the corneal fluorescein staining, drops of 1% fluorescein sodium was prepared on fluorescein paper strips (Madhu Instruments Pvt. Ltd., Okhla Industrial Area, India), after which 5 μL solution was dropped into the conjunctival sac. The eyes were examined and graded under a slit lamp SL-15 with a cobalt blue filter (Kowa company, Ltd., Tokyo, Japan). According to the grading standards of the corneal fluorescein staining from a phase 3 clinical trial for the FDA approved dry eye drug, Lifitegrast, with slight modifications as follows. The corneal surface is equally divided into 9 regions. A score ranging from 0 to 4 was graded for each region (for a maximum score of 36) with 0.5 point increments, and a lower score indicate a better condition. The scores are: “0” no staining, “I” few or rare punctate lesions, “2” discrete and countable lesions, “3” lesions too numerous to count; and “4” coalescent lesion.

Student t-test was conducted to assess the therapeutic effect if the animals in the 2 groups have comparable scores of corneal staining on Day 7, otherwise, paired t-test was conducted to evaluate the effect of treatment.

Results

A total of 19 male mice were used in this study. Nine mice were assigned to the placebo group and 10 mice anti-miR-328 group. Therefore, the results are based on 18 eyes treated by placebo and 20 eyes treated by anti-miR-328.

Both PBS and anti-miR-328 significantly improved the corneal fluorescein staining in mice with dry eye disease, however, anti-miR-328 therapy appeared to achieve a better outcome (Table 2). The modified staining scores on Day 7 and Day 21 were 31.15 and 17.94 for the PBS group (p=0.0003 by paired t-test, Table 2), and 31.70 and 8.20 for the anti-miR-328 group (p=1.67×10⁹ by paired t-test, Table 2). The improvement by anti-miR-328 was greater than by PBS (p=0.005).

TABLE 2
The results of corneal fluorescein staining
in mouse model of dry eye disease.

| | Score of corneal fluorescein | | P from paired |
|---|---|---|---|
| | staining (m ± SEM) | | t-test within |
| Treatment | Day 7 | Day 21 | each group |

Modified staining score for mouse eyes

| | | | |
|---|---|---|---|
| PBS, n = 18 eyes | 31.15 ± 1.61 | 17.94 ± 2.73 | 0.0003 |
| Anti-miR-328, n = 20 | 31.70 ± 1.09 | 8.20 ± 2.18 | 1.67 × 10⁻⁹ |
| eyes | | | |

### Example 3. In Vivo Study of Rabbit Model to Evaluate the Efficacy of Anti-miR-328 Oligonucleotides in Treating BAC-Induced Dry Eye Disease

Rabbit Model of Dry Eye Disease

Materials and Methods

The pigmented Rex rabbits were used in the study and the rabbits were randomly assigned to the PBS treatment or anti-miR-328 treatment group before the induction of dry eye disease by BAC. The dry eye disease was induced by ocular instillation of 0.15% BAC of 20 μL (Sigma-Aldrich, St. Louis, MO, USA) twice per day (9:00 and 17:00) for 1 week. On Day 8, the rabbits started to receive PBS or anti-miR-328 treatment twice per day for 2 weeks while BAC instillation remained twice per day in these two weeks. The order of treatment in these 2 weeks was ocular instillation of PBS or anti-miR-328 first, followed by BAC instillation with a 10-min interval.

Outcome Assessment

The therapeutic effect was evaluated after 2-week anti-miR-328/PBS treatment as described above. Furthermore, a second grading called “Ocular total score” was used to assess the therapeutic effect on rabbit eyes. “Ocular total score” was modified from the Cornea and Contact Lens Research Unit (CCLRU) grading scale and a published guideline (see Takamura E. et al. Japanese guidelines for allergic conjunctival diseases 2017. Allergol Int. 2017; 66:220-229; and Su G., Wei Z., Wang L., et al. Evaluation of Toluidine Blue-Mediated Photodynamic Therapy for Experimental Bacterial Keratitis in Rabbits. Transl Vis Sci Technol. 2020; 9:13), and the scales were based on the following 4 domains: limbal hyperemia, bulbar conjunctival hyperemia, tarsal conjunctiva hyperemia and keratitis. There are 4 severity levels (from 0-3) for the first 3 domains, and 5 severity levels (from 0-4) for keratitis. The rabbit eyes were also used for histology examinations including corneas and Meibomian glands.

Histological Analysis

On Day 21, the rabbits were euthanized humanely, after which the right eye globes were enucleated and immersed in Davidson's fixative (20 ml of 37% formalin, 100 ml of glacial acetic acid, 350 ml of 95% alcohol and 530 ml of water). The tissues were fixed for 48 hours, then washed in tap water before being transferred to 10% neutral buffered formalin for storage prior to trimming and processing. In addition, the eye adnexa were removed and immediately fixed in 10% buffered formaldehyde solution for 24 hours. Subsequently, collected samples (cornea, conjunctiva, and Meibomian gland) were then dehydrated with a gradient series of ethanol and embedded in paraffin. After microtome sectioning, embedded tissue blocks were stained with hematoxylin and eosin (H&E) for histological examination. Histological images of all specimens were observed under an automatic digital slide scanner (Pannoramic mini II, 3dhistech Ltd., Budapest, Hungary), and visualized as well as their measured by using the CaseViewer software (https://www.3dhistech.com/caseviewer).

The TUNEL assay was performed to assess apoptosis in the corneal epithelium and stroma. The TUNEL assay was performed using the in situ Cell Death Detection Kit, POD (number 11684 817910) according to the manufacturer's instructions (Roche. Indianapolis, IN). The apoptotic cells were counted in three randomly selected fields under the 40× magnitude microscopic field.

Hyperkeratosis in the orifices of Meibomian glands in the upper lids were evaluated. The percentage of obstruction by hyperkeratosis in each orifice on a histology slide of 1200 μm was calculated. The mean of obstruction from all orifices in one slide was used to indicate the therapeutic effect on this specific eye.

Conjunctival Impression Cytology

Conjunctival impression cytology specimens were collected on Days 0, 7, 14, and 21. After instilling 0.5% Alcaine and wiping away the excessive fluid from the eye, half-acircular piece of nitrocellulose filter paper (Toyo Roshi Kaisha, Ltd., Japan) with a diameter of 5.5 mm was placed on the superior bulbar conjunctiva. The filter paper was held in place for 1 min via slight pressure and was then peeled off from the eye and immediately fixed with 10% neutral buffered formalin. Then, the papers were stained with the PAS kit following the manufacturer's instructions (PAS-2-IFU, ScyTek Laboratories, Inc., Logan, U.S.A.). PAS reagent was used to stain the tissues, and the number of goblet cells was counted under a microscope at a magnification of 400. After staining, the density of goblet cells was quantified.

### Example 4. Corneal Abrasion by Physical Damage and Evaluation of the Efficacy of Anti-miR-328 Oligonucleotides

Mouse Model of Corneal Abrasion

Materials and Methods

C57BL/6 mice were first under anesthesia and Alcaine was instilled to both eyes to further reduce any subsequent discomfort during physical damage to the corneas. To make an abrasion, use the cleaned ocular Algerbrush. Open one eye at a time by holding the eyelids separately with fingers. Then firmly touch Algerbrush to the cornea and move the instrument back and forth as well as sideways on the ocular surface to induce corneal abrasion. Corneal fluorescent staining was performed after the procedure, and corneal fluorescent staining was performed daily afterward. The left eyes were treated with PBS and right eyes anti-miR-328 twice per day (9 am and 5 μm).

Outcome assessment is by the size of fluorescein stained cornea as described above.

Results

BAC Effect on miR-328

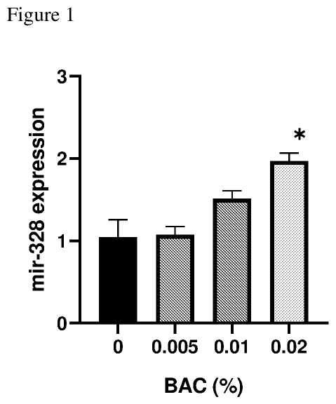

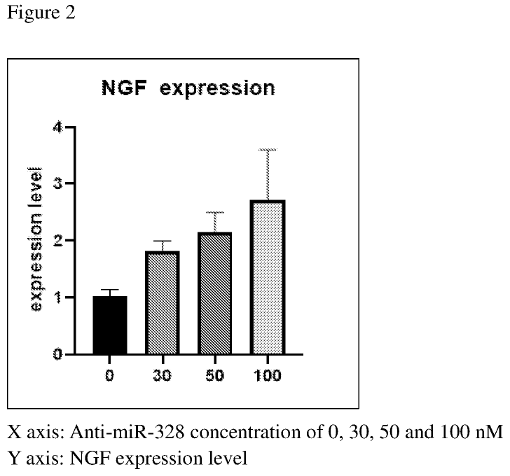

The rabbit corneal cell line, SIRC, was treated with different concentration of BAC. miR-328 expression had dose-dependently increase (FIG. 1). The data, mean±SEM, were from 3 independent experiments. The treatment of anti-miR-328 to SIRC exposed to BAC caused an increase of NGF expression (FIG. 2).

Corneal Staining

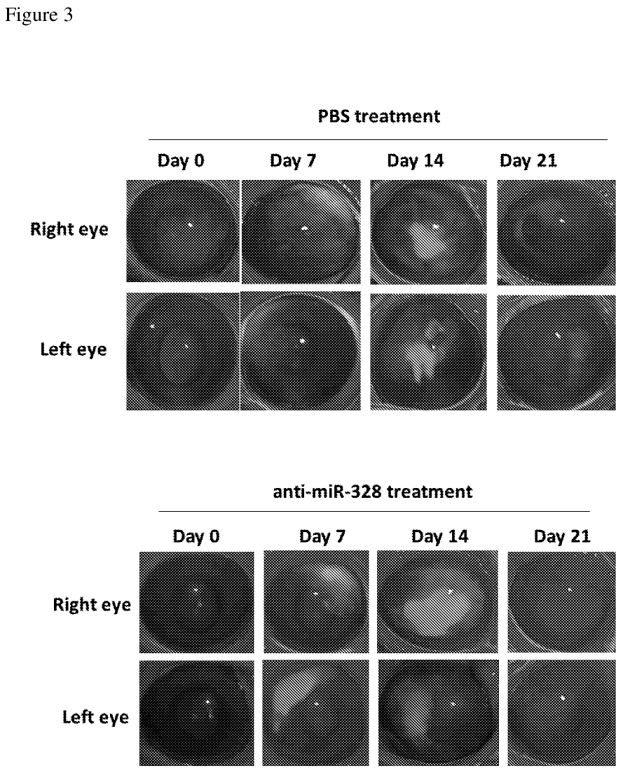

A total of 40 eyes received PBS treatment and 42 eyes received anti-miR-328 treatment. Anti-miR-328 was shown to exert a therapeutic effect on reducing corneal staining on rabbit eyes (FIG. 3). The anti-miR-328 eye drops significantly reduced the modified staining scores (p=0.038 by paired t-test, Table 3) after 2-week treatment, but PBS had no effect on the corneal staining (p=0.699 by paired t-test, Table 3). A similar pattern was observed when the data were analyzed by the ocular total score. The mean of the this score significantly got worse in the PBS group (p=7.4×10⁻⁸ by paired t-test, Table 3), while the anti-miR-328 group had marginal improvement (p=0.053 by paired t-test, Table 3).

TABLE 3
The results of corneal fluorescein staining
in rabbits with dry eye disease.

| | Score of corneal fluorescein | | P from paired |
|---|---|---|---|
| | staining (m ± SEM) | | t-test within |
| Treatment | Day 7 | Day 21 | each group |

Modified staining score for rabbit eyes

| | | | |
|---|---|---|---|
| PBS, n = 40 eyes | 12.60 ± 1.37 | 11.98 ± 1.49 | 0.699 |
| Anti-miR-328, n = 42 | 11.45 ± 1.31 | 8.88 ± 1.68 | 0.038 |
| eyes | | | |

Ocular total score for rabbit eyes

| | | | |
|---|---|---|---|
| PBS, n = 40 eyes | 5.35 ± 0.25 | 8.13 ± 0.30 | *7.4 × 10⁻⁸ |
| Anti-miR-328, n = 42 | 6.57 ± 0.30 | 5.62 ± 0.34 | 0.053 |
| eyes | | | |

*Significantly worse in the PBS group

Thickness of Cornea

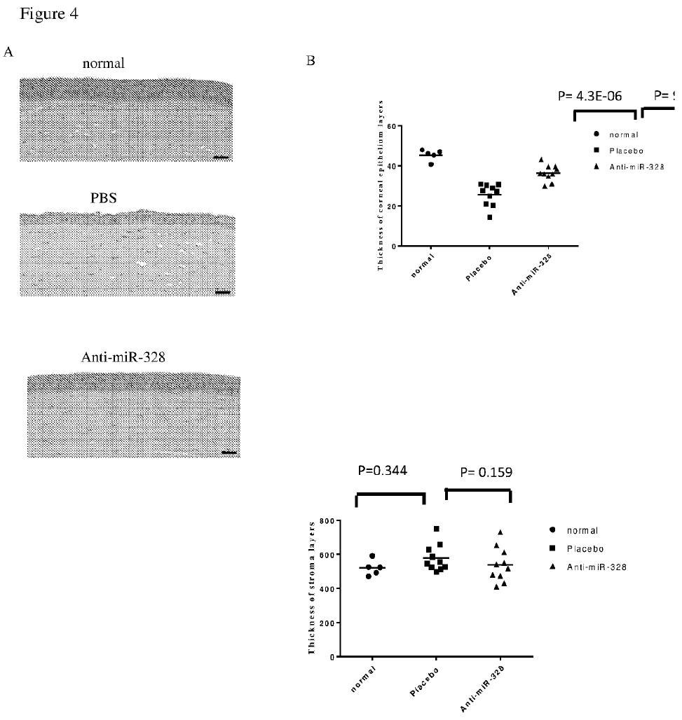

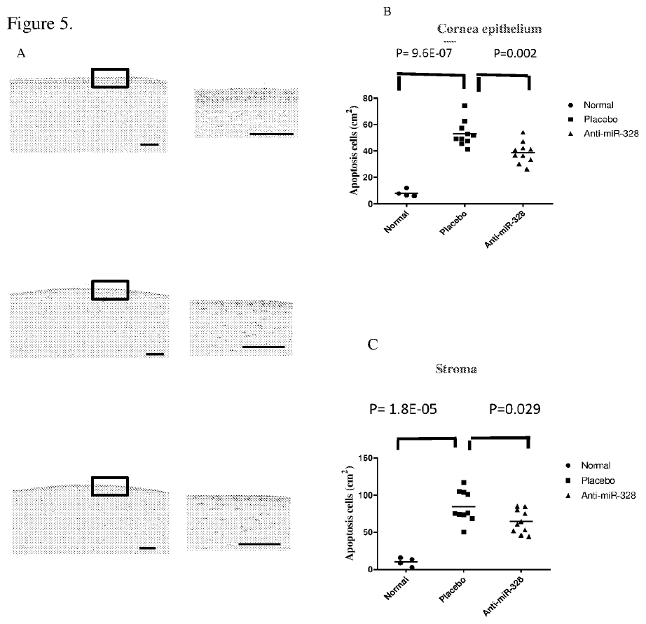

The mean thickness of corneal epithelium was significantly different between the rabbits with dry eye disease treated by anti-miR-328 or PBS eye drops (36.4±1.2 μm vs 25.6±1.7 μm, p=9.4×10⁻⁵), while it was 45.4±1.2 μm for normal rabbits (FIG. 4). The difference of stromal thickness was not statistically significant (p=0.34) between the PBS group and anti-miR-328 group (578.8±25.0 μm vs 539.5±31.8 μm, FIG. 4). The stromal thickness was 521.2±20.4 μm for normal rabbit eyes. The TUNEL assay showed more apoptotic cells in the PBS group than in the anti-miR-328 group in both corneal epithelium (53±3 cells vs 39±3 cells, P=0.002) and stroma (8417 cells vs 65±5 cells, P=0.029) (FIG. 5).

Meibomian Gland Histology

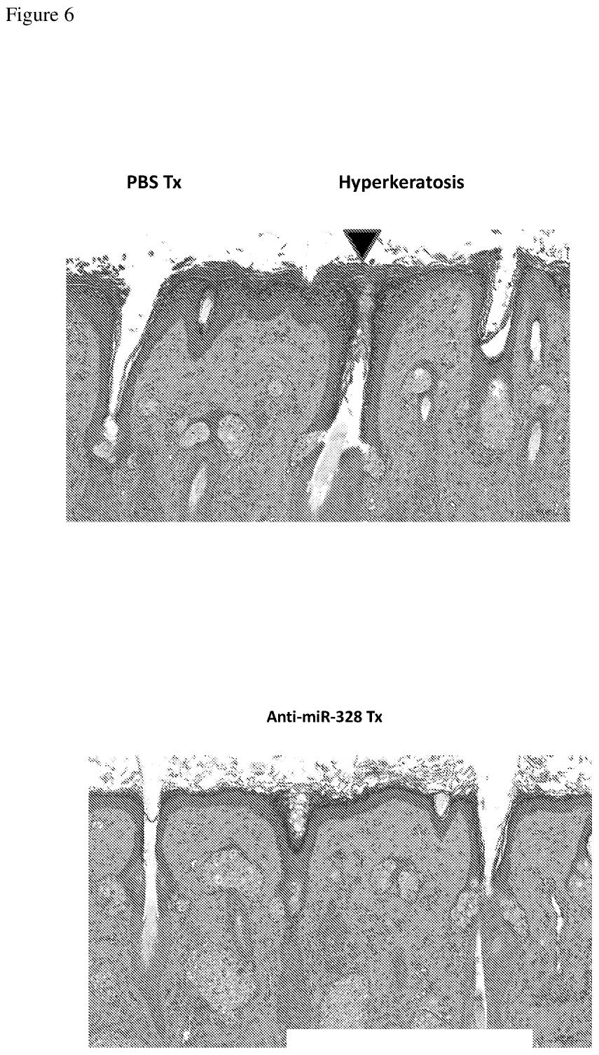

The histology shows that anti-miR-328 group had a lower mean percentage of obstruction than that in the PBS group (56.4%±7.59% vs 78.5%±8.17% vs p=0.059) (FIG. 6), although the difference did not reach a significant level of 0.05.

Conjunctival Goblet Cells

The density of conjunctival goblet cells in the anti-miR-328 group was significantly higher than that in the PBS group (26 vs 19 cell/mm², p=0.005). This finding is consistent with the previous report of a low density of goblet cells in the dry eye disease.

Dose-Dependent Effect

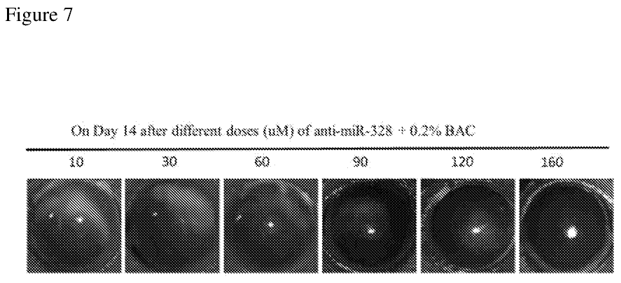

To find the dose to reach the maximal therapeutic effect on dry eye disease, we tested the following anti-miR-328 doses: 10, 30, 60, 90, 120 and 160 μM in mice with dry eye disease. The data indicated a dose-dependent effect on reducing corneal staining and the eyes treated with the dose of 160 μM of anti-miR-328 contained eye drop almost had no fluorescent staining on Day 14 (FIG. 7). Accordingly, anti-miR-328 at 160 μM may be the optimal dose for treating dry eye disease in mice.

Corneal Abrasion by Physical Damage and Evaluation of the Efficacy of Anti-miR-328 Oligonucleotides

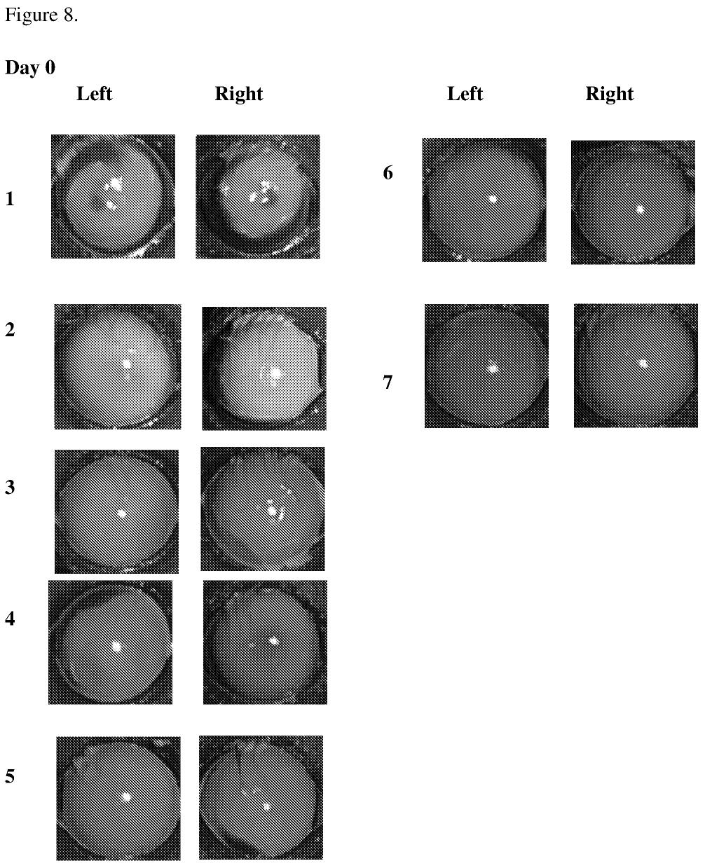

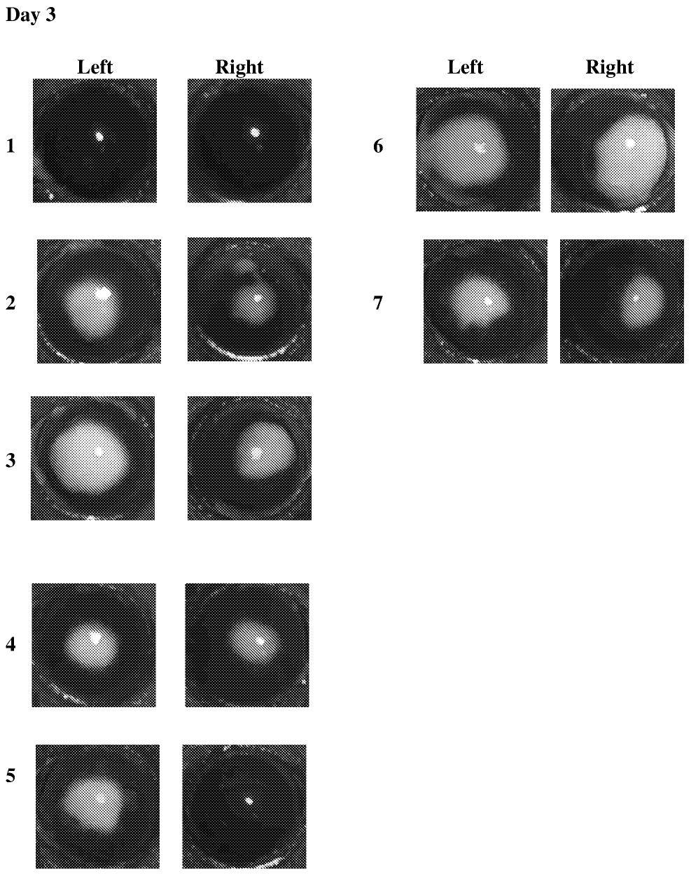

To demonstrate the effect of anti-miR-328 on corneal repairment, the mouse cornea was damage by Algerbrush. After abrasion, the corneal staining shows that the fluorescein was observed on the entire cornea on Day 0, which indicates a complete corneal abrasion (FIG. 8). However, anti-miR-328-treated eyes have faster and better recovery than the PBS-treated eyes on Day 3 (FIG. 8).

The invention, and the manner and process of making and using it, are now described in such full, clear, concise, and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude the specification.

What is claimed is:

1. A method of treating ocular surface damage caused by an eye disease or eye injury in a subject, comprising administering to said subject a pharmaceutical composition comprising a therapeutically effective amount of microRNA-238 antagonist.

2. The method of claim 1, wherein the eye disease or eye injury is any of dry eye disease, chemical or physical injury, infection, neurosensory abnormalities and unspecified etiologies.

3. The method of claim 1, wherein the microRNA-238 antagonist is an anti-miR-328 oligonucleotide comprises an oligonucleotide sequence complementary to miR-238, or a precursor thereof.

4. The method of claim 2, wherein the anti-miR-328 oligonucleotide ranges from 15 to 22 nucleotides in length.

5. The method of claim 2, wherein the anti-miR-328 oligonucleotide is 16 or 17 nucleotides in length.

6. The method of claim 3, wherein the anti-miR-328 oligonucleotide is consisted of SEQ ID NO: 3 or SEQ ID NO:4.

7. The method of claim 5, wherein the anti-miR-328 oligonucleotide is consisted of SEQ ID NO: 3.

8. The formulation of claim 5 wherein the anti-miR-328 oligonucleotide is at a concentration of 1-500 or 10-160 uM.

9. The method of claim 1, wherein ocular surface damage is caused by dry eye disease.

10. The method of claim 1, wherein ocular surface damage is caused by neurotrophic keratitis.

11. The method of claim 1, wherein ocular surface damage is corneal abrasion due to physical injury.

12. The method of claim 1, wherein ocular surface damage is caused by chemical injury.

13. The method of claim 1, wherein ocular surface damage is caused by Meibomian gland dysfunction.

14. The method of claim 1, wherein the pharmaceutical composition is administered to the eye topically.

15. The method of claim 1, wherein the pharmaceutical composition is administered in the form of eye drops.
